(19) United States
(12) Patent Application Publication
(10) Pub. No.: US 2024/0377378 A1
(43) Pub. Date: Nov. 14, 2024

(54) TRACER METHODS FOR THE DETERMINATION OF SORBENT CONTENT IN SOILS

(71) Applicant: Regenesis Bioremediation Products, San Clemente, CA (US)

(72) Inventors: Anh Le-Tuan Pham, San Clemente, CA (US); Neil R. Thomson, San Clemente, CA (US); Alannah R. Taylor, San Clemente, CA (US); Paul R. Erickson, San Clemente, CA (US); Jeremy Birnstingl, San Clemente, CA (US)

(21) Appl. No.: 18/659,147

(22) Filed: May 9, 2024

Related U.S. Application Data
(60) Provisional application No. 63/501,574, filed on May 11, 2023.

(51) Int. Cl. G01N 33/24

(52) U.S. Cl. CPC G01N 33/24

(57) ABSTRACT

Methods are disclosed for determining the amount of sorbents in a soil sample. A known amount of primary tracer can be applied to the sample, causing the primary tracer to start partitioning on the sorbent material. The amount of primary tracer which has partitioned onto the sorbents in this manner will correlate with the amount of sorbents present in the sample, and by measuring this amount of partitioned primary tracer, one can calculate the amount of sorbents present. These methods can be performed on ex situ samples taken from a remedial site or on an in situ sample via the application and measurement methodologies being field-deployable.

### CROSS-REFERENCE TO RELATED APPLICATIONS

This application relates to and claims the benefit of U.S. Provisional Application No. 63/501,574 filed May 11, 2023, and entitled “TRACER METHODS FOR THE DETERMINATION OF SORBENT CONTENT IN SOILS”, the entire disclosure of which is hereby wholly incorporated by reference.

### STATEMENT RE: FEDERALLY SPONSORED RESEARCH/DEVELOPMENT

This invention was made with Government support under a U.S. Department of Defense funded Strategic Environmental Research and Development Program ER21-3959. The Government has certain rights in the invention.

### BACKGROUND

### 1. Field of Invention

The present application relates to determining the amount of sorbents present in an underground remedial site. More specifically, the present application relates to the application of a known amount of a tracer substance to a sample from a remedial site containing sorbents and allowing the tracer to quantitatively partition onto those sorbents in order to determine the amount of sorbents in that sample.

### 2. Related Art

The production, handling and use of chemicals has inadvertently led to their release and undesirable distribution within the environment. Soil, and groundwater are media commonly requiring intervention to remove these unwanted chemicals or mitigate their harmful effects. Several methods exist to support such remediation efforts, including removal and relocation of heavily impacted soils (landfilling), engineered systems to strip out contaminants from the aqueous or vapor phase, and approaches that rely on adding materials to impacted soils or groundwater to reduce contaminant concentration and/or mobility. These last approaches are collectively known as in situ remediation.

One in situ technique growing in popularity involves emplacing adsorptive materials within impacted soils and groundwater; compositions and methods related to this technique are disclosed in U.S. Pat. No. 7,585,132 entitled “METHOD FOR REMEDIATING A CONTAMINATED SITE”, U.S. Pat. No. 9,770,743 entitled “COLLOIDAL AGENTS FOR AQUIFER REMEDIATION”, U.S. Pat. No. 9,776,898 entitled “TREATMENT OF AQUIFER MATRIX BACK DIFFUSION”, and U.S. Pat. No. 10,512,957 entitled “COLLOIDAL AGENTS FOR AQUIFER AND METALS REMEDIATION”, in which the entire disclosures of each are wholly incorporated herein by reference. Adding adsorptive materials to soils both above and below an aquifer (or groundwater-bearing zone) can be of remedial value, as this can drastically increase the partitioning of contaminants out of the dissolved, mobile aqueous phase and into the solid matrix of the natural system. There are a variety of sorbents that may be used for this purpose, some of which include activated carbon, clays, organically-modified clays, zeolites, and particles of cyclodextrin-based polymers. For emplacement within an aquifer, sorbents are typically injected as a suspension of solids dispersed in a carrier fluid. Sorbents of differing particle sizes (typically measured as the diameter of these particles) may be injected, ranging from granule (0.5-5 mm in diameter), powdered (hundredths to tenths of mm), and low micron to colloidal (0.1-10 μm).

An injection method may be used to deliver and disperse these sorbents in the subsurface, and the sorbent's particle size can lend itself to a particular injection method. Particles in the granular and powdered size ranges, being too large to travel through existing pore spaces, usually require high pressure (>100 psi injection pressure) application to disrupt (fracture) the soil structure, whereas low micron to colloidal particles may be injected with low pressure (<100 psi injection pressure) techniques to leave the intact soils relatively undisturbed. When using any combination of the above application methods and sorbent sizes, uncertainty may arise regarding the distribution of the sorbents in the subsurface. Heterogeneity of flow for the injected substance during injection and sorbent transport by groundwater flow after injection are examples of phenomenon that can contribute to this uncertainty. Knowing the presence, location, and concentration of these sorbents within soils is critical for proper construction and performance validation of this class of remediation efforts.

Current methods available for determining sorbent concentrations in soil are limited and unsatisfactory. Existing methods include visual inspection of a soil sample (i.e., comparison with a color chart such as the Munsell color chart), combustion of a soil sample, or other total organic/inorganic carbon-based analyses for activated carbon sorbents. While some of these methods can provide reasonably accurate quantitative data on sorbent concentration, they require analysis after collection in a dedicated laboratory. This adds complexity and precludes informed real-time decision making. The development of a field deployable method would enable real-time decision-making about sorbent and supplementary additive placement and faster delineation of the sorbent following injection.

### BRIEF SUMMARY

To solve these and other problems, methods are contemplated herein for determining the concentration of a sorbent within a treated sample (i.e., a treated soil matrix) by the addition of a primary tracer substance that could be selectively adsorbed by the sorbent with minimal interference by the sample. This uptake by the sorbent could be quantitatively determined by an analytical method, which could be field-deployable when the sample is in situ. This method may also be of use in alternative settings such as soil conditioning with biochar in agricultural applications, the stabilization/solidification of contaminated soils, or sand filter sorbent determination in water treatment applications, for example. The present disclosure outlines these approaches and provides working examples of how this invention could be put into practice.

A method of measuring an amount of sorbents in a sample may comprise the steps of applying a known amount of a primary tracer to the sample, allowing an amount of the primary tracer to partition onto the sorbents, measuring the extent of this partitioning process (e.g., by quantifying the amount of the primary tracer that partitioned onto the sorbents or quantifying the amount of primary tracer which have not partitioned onto the sorbents), and, based on that measurement, calculating the amount of sorbents in the sample. The process of the primary tracer partitioning upon the sorbent can be allowed to reach an equilibrium. Since the extent to which a primary tracer partitions onto sorbents will correlate with the amount of sorbents present in the sample, the amount of sorbents present may be extrapolated from a measurement of the extent of this process.

The primary tracer can comprise dyes, inorganic substances like metal ions, inorganic complexes, organometallic compounds, and combinations thereof. Specific examples of primary tracers which have been successfully tested include Orange G, Rhodamine B, and Congo Red. The sorbents could include activated carbons, biochar, ion exchange resins, clays, organo and inorgano-organo-modified clays, cyclodextrin based media, chitin-based sorbents, and combinations thereof. These sorbents could have a D90 value ranging from 0.01 μm to 0.5 cm, more preferably 0.5 μm to 1 mm, and most preferably 0.5 μm to 10 μm.

The sample could be in situ during the steps of applying the primary tracer, allowing the primary tracer to partition onto the sample, and/or measuring the amount of primary tracer which has partitioned onto the sample. In the last case, a measuring device, such as a rod-mounted instrument, could at least partially be in situ while taking a measurement. The measuring device could comprise a visual probe, a camera, a refractometer, or combinations thereof. To help in applying a primary tracer to an in situ sample of soil, the method could further comprise a step of creating an access point, through which the primary tracer may gain access to the sample.

The sample could be ex situ when applying the primary tracer, allowing the primary tracer to partition onto the sample, and/or measuring the amount of primary tracer which has partitioned onto the sample. In this respect, the sample could be placed in a container (like a vial or bottle).

The primary tracer could be formulated as a solution with a solvent, preferably water. For in situ embodiments, the solution could be applied to the sample, whereafter at least some of the solution could be removed from the sample to measure a change in primary tracer concentration ex situ. Alternatively, the solution could remain in situ while the same type of measurement could take place. The aforementioned rod-mounted instrument may perform said measurement. The measurement technique used could comprise spectrometry, electrochemical measurements, radiochemical measurements, luminescence measurements, colorimetric measurements, or combinations thereof.

A secondary tracer could also be applied to the sample before, after, or alongside the primary tracer. If the primary tracer is formulated as a solution, the secondary tracer can be included in that solution. The secondary tracer could have a reduced affinity for the sample, the sorbents, or both relative to the primary tracer. This secondary tracer could act as a baseline for the primary tracer, in that both types of tracers could experience dilution effects or loss from recovery percentages. By virtue of the secondary tracer having a reduced affinity, one could measure a change in the secondary tracer's concentration to find how much primary tracer was lost to these same effects and extrapolate how much primary tracer had partitioned onto the sorbents in the sample.

All of these embodiments are contemplated to be within the scope of this disclosure. These and other embodiments will become readily apparent to those skilled in the art from the following detailed description of the preferred embodiments, the disclosure not being limited to any particular preferred embodiment.

### BRIEF DESCRIPTION OF THE DRAWINGS

These and other features and advantages of the various embodiments disclosed herein will be better understood with respect to the following description and drawings, in which:

FIG. 1 shows the results of experiments testing the effectiveness Orange G as a primary tracer for polymer-free colloidal activated carbon in various samples;

FIG. 2 shows the results of experiments testing the effectiveness Orange G as a primary tracer for polymer-stabilized colloidal activated carbon in various samples; and

FIG. 3 shows the results of experiments testing the effectiveness Orange G as a primary tracer for powdered activated carbon in various samples.

### DETAILED DESCRIPTION

Disclosed herein are methods for quantifying sorbents in a soil matrix by addition of a primary tracer substance to a sample-sorbent mixture that may quantitatively partition onto the sorbent. Methods may involve the quantitative application of a known amount of a primary tracer to a known or later determined amount of a sample (i.e., soil). Ideally, this primary tracer would have a minimal or known affinity for said sample and be capable of partitioning onto the sorbents within the sample matrix. Once this partitioning process between the primary tracer and sorbents reaches an equilibrium, a measurement may be taken to determine an extent thereof (e.g., measuring the amount of primary tracer which has partitioned onto the sorbents or measuring the amount of primary tracer remaining in a solution applied to the sample). A correlation may exist between the amount of primary tracer that has partitioned in this manner and the amount of sorbents in the sample; as such comparing this measured value with the original, known amount of primary tracer applied to the sample may allow one to quantify the amount of sorbents in the sample. In certain embodiments, a secondary tracer, which could have a reduced affinity for the sample, sorbent, or both relative to the primary tracer, may be added to the sample-sorbent mixture alongside the primary tracer. By comparing the quantity of secondary tracer added to the sample with a later measurement of secondary tracer remaining in the sample, one may gain insight into dilution effects and/or recovery percentages the primary tracer may experience, which can help to accurately determine how much of the primary tracer has partitioned onto the sorbents. The tracer(s) and measurement methodologies may be performed on an ex situ sample of soil or they may, advantageously, be field-deployable, allowing one to apply tracers to an in situ soil matrix containing sorbents and quantify those sorbents, affording great convenience for users at a remedial site.

This description sets forth the functions and features in connection with the illustrated embodiments. It is to be understood, however, that the same or equivalent functions may be accomplished by different embodiments that are also intended to be encompassed within the scope of the present disclosure. It is further understood that the use of relational terms such as primary and secondary and the like are used solely to distinguish one from another entity without necessarily requiring or implying any actual such relationship or order between such entities.

For the purposes described in the background, sorbents may be deposited into samples like soils in various ranges depending on the objectives of the remediation or alternative efforts. In this disclosure, the sorbent concentration may be defined by the dry weight percentage of the added sorbent to the sample matrix. For example, within a treated area containing 1000 kg of dry weight sample that received a sorbent treatment with the total mass of such sorbents totaling 1 kg, the weight percent would be 0.1% by wt. Note that for the definition given the additional weight to the final system, the weight added by the addition of sorbents, is omitted, but a more detailed determination is also acceptable in which the final mass of the sample-sorbent combination is used in the calculation. Other ways to express this same mentioned sorbent loading include 1000 mg sorbent/kg sample or 1000 ppm and may be used later in this disclosure.

The concentration determination methods with the use of tracers described herein can be designed to quantify sorbent concentrations ranging from 0.001% to 10% by wt. In more preferred embodiments, sorbent concentrations can be quantified in a range from 0.05% to 1% by wt., as this range may be more representative of the range of sorbent concentrations deposited during typically subsurface injection applications.

These tracer methods can be used to identify sorbents of various particle sizes in samples. Such sorbent particles identifiable with these methods can have diameters ranging from a D90 value of 0.01 μm up to a D90 value of 0.5 cm. More preferably, the D90 value can range from 0.5 μm up to a D90 value of 1 mm, and in even more preferable embodiments, the D90 value can range from a D90 value of 0.5 μm up to a D90 value of 1 μm. Sorbent particles in this last range are most relevant for remediation injection purposes, but the presently contemplated methods can still prove effective at the broader ranges to encompass other potential applications which could benefit from the methods disclosed herein.

The methods disclosed herein can be applied to any sorbent which possesses one or more qualitative or quantitative properties unlike that of the surrounding sample matrix. These one or more dissimilar properties may induce an increased affinity of the chosen primary tracer to partition onto the sorbent when compared to other species in the surrounding sample matrix. This property can include, but is not strictly limited to, differing hydrophobicity, cationic and/or anionic exchange ability, size/molecular shape exclusion, and combinations thereof. The sorbent behavior described herein may refer to molecular-level adsorption, wherein dissolved molecules become associated with the solid phase (sorbent in this case). The sorbents can be, but they are not limited to, activated carbons, biochar, ion exchange resins, clays, organo and inorgano-organo-modified clays, cyclodextrin-based media, chitin-based sorbents, other biopolymers, and combinations thereof.

A suitable primary tracer may be selected so as to give effectual partitioning results upon the sorbents in the sample matrix (and thus, an accurate measurement of the amount of sorbents present). Any partitioning of the primary tracer onto the sample and other species that may be present in the sample matrix is ideally minimized so that any/the majority of primary tracer applied to the soil can be attributed to adsorption onto any sorbents that may be present. If a significant affinity between the primary tracer and a non-sorbent species exists, the amount of primary tracer which partitions to non-sorbent species may be known or predictable such that one can account for this effect when measuring the amount of primary tracer which partitioned onto the sorbents. In this respect, experiments may be performed (e.g., one on a sample free of sorbents and another on the same type of sample having a known amount of sorbents therein) to quantify how a certain tracer may partition onto the non-sorbent species present in a particular sample being tested. Once this affinity of the primary tracer for non-sorbent species in a certain sample has been found, one can, when testing the same type of sample (having similar non-sorbent species) with an unknown amount of sorbents therein, subtract the amount of primary tracer which would have partitioned onto the non-sorbent species to find an accurate value for how much primary tracer has partitioned onto the sorbents.

The primary tracer would preferably have little to no reactivity with the species of a sample matrix or the sorbent, be generally stable, and not degrade by hydrolysis or other abiotic means on the hour timescale. Primary tracers will also ideally be nonhazardous and acceptable in low amounts for release into environmental systems, particularly in the case of the in situ embodiments. Additionally, primary tracers which lend themselves to convenient quantitative analysis techniques that can be performed rapidly and are field deployable are preferable. For example, a primary tracer with an identifiable absorption feature which is intense enough (extinction coefficient above 1,500 M⁻¹ cm⁻¹) within the common range of commercial UV/vis/near-IR spectrophotometers (200-900 nm in wavelength) and/or a primary tracer which fluoresces with a reasonable intensity within a similar range (300-900 nm in wavelength) may be particularly preferred for spectroscopic analyses.

The primary tracer may include, but is not limited to, organic compounds like dyes (including rhodamine dyes and fluorescein), inorganic substances like metal ions, inorganic complexes, organometallic compounds, and combinations thereof. Examples of a primary tracers that have been successfully tested with the methods disclosed herein include Orange G, Rhodamine B, and Congo Red, with Orange G being found to have the most effective results. Working embodiments and properties of effective primary tracers will be discussed later in this disclosure.

A secondary tracer may also be chosen having similar properties to the primary tracer. In this respect, the secondary tracer may simply be another tracer applied to the soil to measure the amount of sorbents in the sample being tested. In other embodiments, however, the secondary tracer chosen may have a reduced affinity for the sample, sorbents, or both relative to the primary tracer. By recording the amount of secondary tracer applied to a sample and thereafter measuring the amount of secondary tracer remaining in the sample, one may be informed of dilution effects and/or recovery losses that the primary tracer could experience. Thus, the secondary tracer, having minimal affinity for the species it is exposed to (particularly the sorbents), could act as a baseline for the primary tracer such that any reduction in a measurement for the amount of secondary tracer can be proportionate to a reduction of the primary tracer. One could thus account for this difference when determining the actual amount of primary tracer which has partitioned onto the sorbents of a sample. The number of tracers applied to a sample need not be limited to one or two; therefore, a tertiary tracer, quaternary tracer, etc., can also be applied to that sample.

It may be desirable to calibrate the partitioning process of the primary tracer to the sorbent such that the degree of partitioning may be sufficiently quantified in the actual samples to be tested or in a test sample of suspected similar composition. Calibration can involve applying a known amount of target sorbent to a known amount of sample for a series of samples. The samples, calibration range, primary tracer type, and detection method may fall within any of the variants described herein. This may be done for both in situ and ex situ embodiments. An important aspect to also be evaluated within method calibration is proper equilibration times required for particular primary tracer-sorbent-sample combinations. Effort can be made to ensure that this required time is known and given, or at least the time used standardized for a particular variant of the method. Such calibration will be elaborated upon later in this disclosure.

Tracers may be applied to a sample containing sorbents. Application to a sample may be performed by injection. A primary tracer, optionally including a secondary tracer and any further tracers, may be formulated as a solution with a solvent, in which case the primary tracer's original concentration in the solution may range from 1 μg/L to 500 g/L. In most instances, the preferred solvent will be water. It may be desirable to include additives such as cosolvents or solution conditioners (e.g., pH or ionic strength modifiers, flocculants), particularly in ex situ embodiments. Organic cosolvents could include alcohols and other miscible or highly water-soluble compounds, which could modify the sorption affinity of the primary tracers for the sorbent, sample, or both. Reasonable addition rates for any of the abovementioned solution conditioners or cosolvents may range from 0.001-50% of the solution volume.

For ex situ embodiments, the method can be conducted in a container (e.g., a vial or bottle) containing a known mass of the sample to be tested (which could be a soil sample taken from a remedial site). A known mass of solvent, primary tracer, and any optional additional tracers can be applied to the sample. Preferably, all components would be applied to the sample simultaneously as a solution, but individual components/certain combinations of components may be applied to the sample at different times. If the primary tracer is formulated as a solution, the concentration of the primary tracer can be known prior to application to the soil sample. The liquid volume applied may range from 0.5 mL-4000 mL and be mixed with a soil mass ranging from 0.1 g to 1000 g. The primary tracer may be allowed to partition onto the sorbents present in the sample, ideally such that this partitioning reaches an equilibrium. One may thereafter measure the amount of primary tracer present in the solution (such as with a mass or concentration measurement), subtract this value from the original known mass/concentration of primary tracer prior to the application on the sample to find an amount of “missing” primary tracer, account for any significant affinity the primary tracer may have for non-sorbent species in the sample and/or dilution effects by subtracting accordingly to find an amount of tracer which has partitioned onto the sorbents, and based on that value, determine the amount of primary tracer that has partitioned onto the sorbents.

In situ embodiments may follow a similar procedure in that a known amount (i.e., mass, concentration) of a primary tracer can be applied to an in situ soil sample. The primary tracer can also be formulated as a solution with a solvent to be applied to the soil sample. A couple of application methodologies may be used to apply a tracer to an in situ soil sample, including a push-pull mechanism and a rod-mounted implementation. For the push-pull approach, a drilling machine like a Geoprobe® may mechanically drive rods into soil to reach an area to be tested. This may create an access point through which the primary tracer can be applied to the sample containing sorbents. Alternatively, pre-existing access points may allow the primary tracers to conveniently access the soil (such as wells, piezometers, etc.), although these pre-existing access points can be expanded with a drilling machine as needed. From the access point(s), the primary tracer can be delivered as solution into the soil, allowed to partition onto the adsorbents (preferably reaching equilibrium), and then withdrawn back out (hence the push-pull mechanism). The withdrawn solution can be measured to determine the amount (i.e., mass, concentration) of primary tracer left in the solution. By subtracting this value from the original known amount of primary tracer applied to the sample one can find a “missing” amount of primary tracer, and by subtracting the amount of primary tracer lost to non-sorbent species and effects (the primary tracer's potential affinity for non-sorbent species in the sample, dilution effects, and/or recovery losses that can arise from removing the solution from the sample), the amount of primary tracer that had partitioned onto the sorbents in the soil can be determined. The volume of liquid applied to the soil for the push-pull mechanism embodiments can range from 1 mL-1000 L. In these embodiments, this volume of tracer could sample a varying mass of soil, which will largely depend on the porosity of the soil or aquifer. To determine this sampled mass and thus sorbent concentration properly, the soil porosity (including potential differences in total porosity and effective porosity) may need to be accounted for. This can be done using standard estimation based on soil type and transmissivity, soil sore collection and direct porosity measurement, or instrumental means like nuclear magnetic resonance direct push logging tools.

The rod-mounted implementation in situ embodiments may also rely upon the creation of and/or use of existing access points to introduce a primary tracer formulated as a solution. The access points can again receive the primary tracer so that it can be allowed to partition onto the sorbents. These embodiments may differ from the push-pull mechanism in that the amount of primary tracer remaining in solution can be measured while the solution remains in situ with a rod-mounted instrument, such as those disclosed in U.S. Pat. No. 10,371,637 entitled “SOIL IMAGING PROBE AND METHOD OF PROCESSING SOIL IMAGE TO DETECT HYDROCARBON CONTAMINATION” and CZ 2008457 entitled “Device for detection and measurement of concentration of fluorescent tracing substance, measuring method and use of such device”, in which the disclosures of both are wholly incorporated herein by reference. One or more measurements can be recorded by the rod-mounted instrument to track the partitioning process between the primary tracer and the adsorbent as it is occurring and determine when an equilibrium has been reached. The measurement device used may determine the amount of primary tracer present in the solution visually (if the measurement device includes, for instance, a visual probe, a camera, and/or a refractometer), quantitatively (wherein the measurement device gathers numerical data), or a combination thereof. Following a measurement at equilibrium, the same series of steps detailed above may be followed to calculate the amount of primary tracer which has partitioned onto the sorbents, although in these embodiments, one may not need to heavily account for recovery losses when compared to the push-pull mechanism. The volume of liquid applied to the soil for the rod-mounted implementation embodiments can range from 1 mL-1000 L.

To measure the primary tracer in a solution, in either in situ or ex situ embodiments, various techniques may be used, including, but not being limited to, spectrometric, electrochemical, radiochemical, luminescence, colorimetric, other commonly employed chemical analytical methods, and combinations thereof. In both ex situ and in situ embodiments, the primary tracer may be allowed to partition onto the sorbents present in the sample until an equilibrium is reached. As would be appreciated by those skilled in the art, this equilibrium need not be a true equilibrium (which could require an infinite timescale), but rather an equilibrium where significant changes no longer occur in the primary tracer's partitioning process upon the sorbents. This could come in the form of a measurement instrument finding no change/a minimal change in concentration of primary tracer in the applied solution, or the partitioning process slowing such that one can determine a range of sorbents present in the sample (as will be discussed later in this disclosure).

The time necessary for the partitioning process between the primary tracer and sorbents to reach an equilibrium may vary based on the primary tracer-sorbent-soil combinations being employed; equilibration times have been found to range from 30 seconds to 48 hours. Sorbents of smaller particle size and/or sample agitation (e.g., shaking the container in ex situ embodiments) may have lesser time to equilibration. Experiments may be performed and data collected to inform one carrying out these methods as to when to expect adequate equilibrium has been achieved, so that they may then measure the amount of primary tracer in solution or remove the solution from the access point(s) in the push-pull mechanism embodiments when that time has elapsed since application. When the time comes to perform a measurement, some of the applied mixture/solution may be isolated. This isolated portion of the solution can be filtered to remove interference from suspended sorbent or sample matrix the isolated portion may contain.

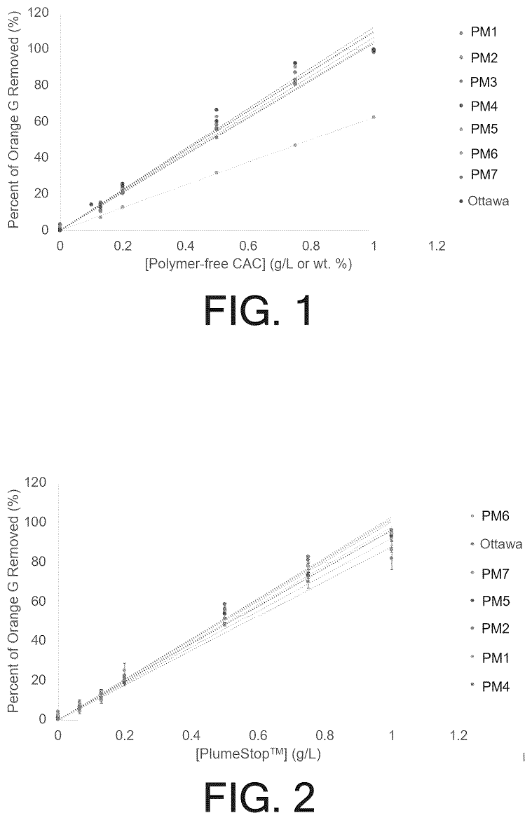

After determining the amount of primary tracer which has partitioned onto the sorbents, or the amount of primary tracer remaining in a solution applied to the sorbents, one may be capable of calculating the amount of sorbents in the sample tested using data from previous experiments showing how the amount of sorbents in the sample would correlated to the degree of partitioning of the primary tracer thereon. Examples of these experiments and data will be discussed in relation to FIGS. 1-3 later in this disclosure. One could calculate a range of values in which the sorbent could be present in the sample tested. How broad or narrow a calculated range is may depend on the sample-sorbent-primary tracer combination of the particular embodiment. For example, in some embodiments the determination of adsorbent content can be done by identifying ranges (windows of concentration, 0-0.2% by wt. vs 0.2%-0.4% by wt. vs. 0.4%-0.6% by wt., etc., for example) or the quantification may be determined by visual inspection alone (to show if sorbent is present above successive thresholds, 0-500 mg sorbent/kg sample, 500-1000 mg sorbent/kg sample), as judged by if the color of a primary tracer is still visible or not when added in aliquots, or alternatively compared to a standard color chart similar to a pool chemistry analysis kit.

The remainder of this disclosure will cover various experimental findings and results relating to various dyes used as primary tracers according to the method described above. As such, the remaining discussions are not intended to be limiting but rather provide examples and experimental evidence. Starting with Table 1, ideal properties of a primary tracer to partition on activated carbon present in an ex situ soil sample are described (four left-most columns), as well as those corresponding properties for three specific primary tracers: Orange G, Rhodamine B, and Congo Red (three right-most columns). It is contemplated, however, that these properties could still be ideal for application upon in situ samples. Spectroscopic techniques were used to quantify the amount of tracer which had partitioned onto the activated carbon. A number of parameters and ranges of those parameters listed in Table 1 have been found to be consistent amongst suitable primary tracers, such as Log(K_{ow})<4, pKₐ<5 or >9, and more. The primary tracers utilized need not be limited to these ranges, but these parameters could be necessary to give effectual results for certain sorbent-sample mixtures. As mentioned earlier, suitable primary tracers will ideally not partition onto the background soil matrix and may have negligible reactivity. The lack of affinity for soils has been found to correspond to having a K_{ow} below 4. The pH sensitivity of the primary tracer may also affect soil and sorbent and affinity, and other characteristics important to the quantification of the primary tracer. Ideally the primary tracer pKₐ will be <5 or >9 to minimize changes to primary tracer's protonation state within the typical environmental pH range. For ex situ embodiments, a pH buffered solution may be used for tracers outside of this pKₐ range while still giving desirable results. The solubility of the primary tracer may also be a property worth considering during primary tracer selection. At environmentally relevant pHs, the primary tracer may ideally be considered generally water soluble. This will correspond to possessing a low Log(K_{ow}), but generally a solubility of 1 g/L or more will be desirable.

TABLE 1

| | | | | Selected Tracers Evaluated | | |
|---|---|---|---|---|---|---|
| | Parameter | Units | Ideal Range or Property | Orange G | Rhodamine B | Congo Red |
| Ideal | Log(K_{ow}) | — | <4 i.e., hydrophilic | −4.56 | 2.28 | 2.63-3.57 |
| Properties | | | or semi-hydrophobic | | | |
| of Primary | pKₐ | — | <5 or >9 i.e., outside | 11.5 | 3.1-3.22 | 4.1 |
| Tracer | | | environmentally relevant | | | |
| | | | pH of 5-9 | | | |
| | Maximum | nm | In the visible range, as to | 480 | 550 | 497 |
| | Absorbance | | be easily measured in the | | | |
| | Wavelength | | field for in situ embodiments. | | | |
| | Extinction | M⁻¹cm⁻¹ | >1,500 | 20,900 | 106,000 | 48,000 |
| | coefficients at | | | (at 480 nm) | (at 545 nm) | (at 500 nm) |
| | the above | | | | | |
| | wavelength | | | | | |
| | Fluorescence | — | Capable of fluorescence, | Limited | Fluorescent | Fluorescent |
| | properties | | to allow for fluorescent | information | solutions are | solutions are |
| | | | measurement | available. | possible. | possible. |
| Additional | Solubility | g/L | Soluble, as to be easily | 50-100 | 20 | 25-116 |
| Data | | | dissolved into an aqueous | Considered | Considered | |
| | | | solution | soluble in water | soluble in water | |
| | Chemical | — | Stable | Stable under | Stable under | Stable under |
| | Stability | | | normal | normal | normal or |
| | | | | conditions | conditions | ambient |
| | | | | | | conditions |
| | Hazard | — | Non-hazardous (Additionally, | Hazard | Hazard | Hazard |
| | Classification | | primary tracers would ideally | Identification: | Identification: | Identification: |
| | | | have a lack of toxicity | Does not meet | Considered | Considered |
| | | | and be safe to handle and | the criteria for | hazardous by | hazardous by |
| | | | dispose of) | hazardous | 2012 OSHA | 2012 OSHA |
| | | | | classification, | Hazard | Hazard |
| | | | | based on the | Communication | Communication |
| | | | | 2012 OSHA | Standard; acute | Standard; |
| | | | | Hazard | oral toxicity | carcinogenicity |
| | | | | Communication | (Category 4), | (Category 1B), |
| | | | | Standard | and Serious eye | and |
| | | | | No WHIMIS | damage/eye | reproductive |
| | | | | labels provided | irritation | toxicity |
| | | | | | (Category 1) | (Category 2) |
| | | | | | WHIMIS labels | WHIMIS label |
| | | | | | of corrosive, | of health |
| | | | | | and less serious | hazard |
| | | | | | health | |
| | | | | | effects/irritant | |

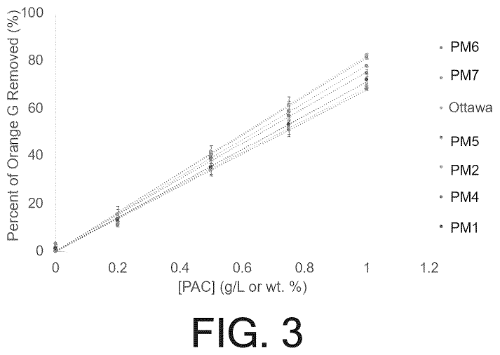

Turning now to FIGS. 1-3, the results of experiments testing the effectiveness Orange G as a primary tracer are shown. 120 trials were performed on various ex situ samples containing activated carbon, with 40 of those trials being done with each of the following types of activated carbon adsorbent: 1-2 μm polymer free colloidal activated carbon (corresponding to FIG. 1), 1-2 μm polymer-stabilized colloidal activated carbon (specifically PlumeStop™, corresponding to FIG. 2), and 10-100 μm powdered activated carbon (corresponding to FIG. 3). Each type of activated carbon adsorbent was mixed with 8 different types of samples. 7 of these samples were from aquifers at field sites across North America, which are designated as PM1, PM2, PM3, PM4, PM5, PM6, and PM7. The last material used was a commercially available sand from an online retailer, which is designated as “Ottawa”. Ottawa sand represents an inert, porous silica media. 100 g/L of each sample was collected and used in their respective trial, and the three types of activated carbon were mixed with each type of soil in the following 5 concentrations: 0.2 g/L, 0.4 g/L, 0.6 g/L, 0.8 g/L, and 1 g/L, thus creating 140 unique trials (8 soil types×3 types of activated carbon×5 concentrations). A solution of Orange G was then added at a concentration of 25 mg/L. Additionally, buffer solutions of 100 mM HCOOH and 100 mM HCOONa were present in each sample to keep the pH in a range from 3.6-6.0, where results have been found to be consistent. The process of Orange G partitioning onto the adsorbents was allowed to reach equilibrium before spectrophotometer measurements were used to calculate the amount of Orange G that was removed by this process.

The results of the experiments with the polymer-free colloidal activated carbon are shown in FIG. 1, in which it can be seen that a strong linear relationship exists between the amount of Orange G removed (the amount of Orange G that has portioned onto the activated carbon) and the amount of activated carbon present. The experiments utilizing the polymer-stabilized colloidal activated carbon (specifically PlumeStop™) are shown in FIG. 2, in which once again it can be seen that a strong linear trend is shown between the increase in activated carbon concentration and the amount of Orange G removed. The results of the last set of trials, in which the larger powdered activated carbon was employed, are shown in FIG. 3. Just like the previous 2 sets of trials, a strong linear relationship between the removal of Orange G and the amount of activated carbon in the soil is present. It can therefore be seen that models can be developed which correlate the measurable amount of primary tracer removed and the amount of sorbent that must be present. One may, as described earlier, still have to take into account other properties of the soil and species present therein which could affect the partitioning of the primary tracer to any sorbents present in the soil, thus creating different levels of correlation between primary tracer removed and amount of adsorbent present, but the experimental embodiments depicted above indicate that regardless of those conditions, a strong correlation may be present.

The above description is given by way of example, and not limitation. Given the above disclosure, one skilled in the art could devise variations that are within the scope and spirit of this disclosure. Further, the various features of the embodiments disclosed herein can be used alone, or in varying combinations with each other and are not intended to be limited to the specific combination described herein. Thus, the scope of the claims is not to be limited by the illustrated embodiments. Additional modifications and improvements of the present disclosure may also be apparent to those of ordinary skill in the art. Thus, the particular combination of parts and steps described and illustrated herein is intended to represent only certain embodiments of the present subject matter and is not intended to serve as limitations of alternative devices and methods within the spirit and scope of this disclosure.

What is claimed is:

1. A method of measuring an amount of sorbents in a sample, the method comprising the steps of:
a) applying a known amount of a primary tracer to the sample;
b) allowing an amount of the primary tracer to partition onto the sorbents;
c) measuring said partitioning in step b); and
d) calculating the amount of sorbents in the sample based on a measurement from step c).

2. The method of claim 1, wherein the primary tracer comprises Orange G, Rhodamine B, Congo Red, or combinations thereof.

3. The method of claim 1, wherein the sorbents comprise activated carbons, biochar, ion exchange resins, clays, organo and inorgano-organo-modified clays, cyclodextrin based media, chitin-based sorbents, or combinations thereof.

4. The method of claim 1, wherein the sorbents have a D90 value ranging from 0.01 μm to 0.5 cm.

5. The method of claim 1, wherein the sample is in situ in step b).

6. The method of claim 5, wherein step c) is based on a measurement with a measuring device, the measuring device at least partially being in situ while taking the measurement.

7. The method of claim 6, wherein the measuring device comprises a visual probe, a camera, a refractometer, or combinations thereof.

8. The method of claim 5, wherein the method further comprises a step of creating an access point; and wherein step a) comprises the access point receiving the known amount of primary tracer.

9. The method of claim 1, wherein the sample is ex situ in step b).

10. The method of claim 1, wherein the primary tracer is formulated as a solution with a solvent.

11. The method of claim 10, wherein after step b), the method further comprises a step of removing at least some of the solution from the sample.

12. The method of claim 11, wherein step c) comprises measuring an amount of primary tracer present in the solution removed from the sample and comparing the measured amount of primary tracer in the solution removed from the sample with the known amount of primary tracer.

13. The method of claim 1, the method further comprises a step of applying a secondary tracer to the sample.

14. The method of claim 13, wherein the secondary tracer has a reduced affinity for the sample, the sorbents, or both relative to the primary tracer.

15. The method of claim 11, wherein the solution further comprises a secondary tracer.

16. The method of claim 15, wherein the secondary tracer has a reduced affinity for the sample, the sorbents, or both relative to the primary tracer; and wherein step c) comprises measuring an amount of the secondary tracer present in the at least some of the solution removed from the sample.

17. The method of claim 1, wherein step c) comprises a measurement methodology, the measurement methodology comprising spectrometry, electrochemical measurements, radiochemical measurements, luminescence measurements, colorimetric measurements, or combinations thereof.
